(19) United States
(12) Patent Application Publication
(10) Pub. No.: US 2026/0294441 A1
(43) Pub. Date: Oct. 1, 2026

(54) EVALUATION OF DELIVERY LOCATIONS FOR A VASCULAR CATHETER FOR TREATMENT OF A TARGET

(71) Applicant: UNIVERSITEIT GENT, Gent (BE)

(72) Inventors: Tim BOMBERNA, Gent (BE); Charlotte DEBBAUT, Gent (BE); Saar VERMIJS, Gent (BE); Jolan HEYSE, Gent (BE)

(21) Appl. No.: 19/490,229

(22) PCT Filed: Jun. 4, 2024
(86) PCT No.: PCT/EP2024/065298

§ 371 Date: Dec. 4, 2025

(30) Foreign Application Priority Data
Jun. 5, 2023 (EP) 23177284.9

(51) Int. Cl. A61B 17/12

(52) U.S. Cl. CPC A61B 17/12186; A61B 17/12109

(57) ABSTRACT

Method for evaluating a plurality of candidate delivery locations (130, a to c) for a delivery of a substance by a vascular catheter (200) for treatment of a target (120) within an organ (110) of a subject comprising: receiving a three-dimensional model obtained from a three-dimensional medical image of at least a part of the organ (110) of the subject, wherein the three-dimensional model includes arterial lumens, and determining from the three-dimensional model a vascular cost, VC_{cdl}, for each candidate delivery location (130, a to c) which is related to an accessibility of the candidate delivery location (130, a to c) by a tip (202) of the vascular catheter (200), wherein the evaluating comprises using the vascular cost for each candidate delivery location (130, a to c) and a likelihood that the substance flowing from the candidate delivery location (130, a to c) towards the target will flow through one or more arterial branches converging with the target and into the target when delivered at the candidate delivery location (130, a to c) by the tip of the vascular catheter.

### FIELD OF THE INVENTION

The present invention is in a field of treating a target by a vascular catheter, more in particular, by embolisation such as transarterial chemoembolisation (TACE) or transarterial radioembolisation (TARE).

### BACKGROUND TO THE INVENTION

In the oncological field, some targets for treatment that are organ tumours cannot be surgically removed and may therefore be treated by embolisation. In embolisation an embolising substance (e.g. microparticles) is injected directly into an artery feeding the tumour, and the embolising substance causes occlusions to the artery at the site of injection or downstream thereof, thereby (partially) preventing supply of blood to the tissue downstream (ideally including the tumour). Embolisation may be combined with delivery of anticancer agents to the tumour (transarterial chemoembolisation (TACE)) or with delivery of radioactive agent to the tumour (transarterial radioembolisation (TARE)). In embolisation, a catheter is inserted via an artery (e.g. femoral artery), navigated into an organ artery, and occluding microparticles (non-active, attached to an anticancer agent, or attached to a radioactive agent) are injected to cause the embolisation downstream of the injection site. In the case of TARE, there is an additional selective irradiation of the target within the organ. In the case of chemoembolisation, there is an additional selective chemotherapeutic effect on the target within the organ.

However, with embolisation procedures there is a wide variation in outcome depending on how embolisation is carried out by the specialist. Currently, the interventional radiologists choose themselves how to implement embolisation based on medical images (e.g. CT/MRI) and based on their experiences or preferences. However, anatomical medical images per se only offer anatomical information, and no insights into other functional aspects.

Where TARE is used, the interventional radiologist may perform a pre-treatment injection of low-dose radioactive particles and take one or more 3D medical images (e.g. PET/SPECT), which provide some information about tumour dose. However, pre-treatment injection differs from actual treatment injection, for instance in particle properties, injection location, timing etc. When the treatment injection dose is injected, the outcome typically differs. Further, pre-treatment injection is not possible when the embolising substance is non-radioactive.

It has been shown that high tumour uptake of embolising substance is instrumental to a positive survival advantage (e.g. Hermann et al., Vascular and Interventional Radiology, 2020, 296:673-684), however, several randomized controlled trials have shown in practice the wide variation in outcome attributed mainly to embolisation being a highly technical procedure whose success depends at least in part on the experience of the medical centre (Sposito and Mazzaferro, HepatoBiliary Surgery and Nutrition, 2018, 7(6) 487-489). It is an aim of the present invention to provide a way to improve and potentially standardise outcome of embolisation procedures.

### SUMMARY OF THE INVENTION

Provided herein is a computer-implemented method for evaluating a plurality of candidate delivery locations (130, a to c) for a delivery of a substance by a vascular catheter (200) for treatment of a target (120) within an organ (110) of a subject comprising:
receiving a three-dimensional model obtained from a three-dimensional medical image of at least a part of the organ (110) of the subject, wherein the three-dimensional model includes arterial lumens,
determining from the three-dimensional model a vascular cost, VC_{cdl}, for each candidate delivery location (130, a to c) which is related to an accessibility of the candidate delivery location (130, a to c) by a tip (202) of the vascular catheter (200), and
determining, for each candidate delivery location (130, a to c) a target dose uncertainty, wherein:
   the target dose uncertainty is a measure of a likelihood that the substance flowing from the candidate delivery location (130, a to c) towards the target will flow through one or more branches leading into the target (200),
   the target dose uncertainty is determined comprising the steps:
      obtaining a segment map (310), wherein the segment map (310) is:
         a transverse cross-section of an arterial lumen in the 3D model at the candidate delivery location (130, a to c);
         divided into a plurality of lumen segments (302, a to e);
   predicting, for each candidate delivery location (130, a to c), a release of microparticles from the segment map (310) in a downstream direction, and a trajectory of each microparticle;
   assigning to each lumen segment (302, a to e), a segment target dose fraction, STDF, value that is an indication of the quantity of microparticles released from that lumen segment (302, a to e) that flow into the target (120) compared with total quantity microparticles released from that lumen segment (302, m to p),
   determining from the STDF values, a cluster target dose fraction, CTDF, wherein a cluster (306) is collection of lumen sections (302, b to e) grouped together to form a shape of a catheter tip (202) at a specific location on the segment map (310), and the CTDF is determined from the STDF values of lumen sections (302, b to e) within that cluster (306),
   determining a plurality of the CTDF values for a sample group, wherein the each CTDF of the sample group is determined from a cluster having a different location on the segment map (310),
   determining from the sample group of CTDF values an uncertainty range for the candidate delivery location (130, a to c),
determining the target dose uncertainty from the uncertainty range, wherein the evaluating comprises using a combination of the vascular cost and the target dose uncertainty for each candidate delivery location (130, a to c).

The uncertainty range may have one of more of:
a maximum CTDF value,
a minimum CTDF value,
a median CTDF value.

Provided herein is a method for evaluating a plurality of candidate delivery locations (130, a to c) for a delivery of a substance by a vascular catheter (200) for treatment of a target (120) within an organ (110) of a subject comprising:
receiving a three-dimensional model obtained from a three-dimensional medical image of at least a part of the organ (110) of the subject, wherein the three-dimensional model includes arterial lumens, and
determining from the three-dimensional model a vascular cost, VC_{cdl}, for each candidate delivery location (130, a to c) which is related to an accessibility of the candidate delivery location (130, a to c) by a tip (202) of the vascular catheter (200), wherein the evaluating comprises using the vascular cost for each candidate delivery location (130, a to c).

The vascular cost of a candidate delivery location (130, a to c) is preferably determined from a set of vascular cost parameters of a portion (P) of the artery lumen, wherein the set of vascular cost parameters comprises at least one of:
minimum diameter, D_{cdl}, of the portion (P) of the artery lumen,
tortuosity, τ_{cdl}, of the portion (P) of the artery lumen,
curvature, κ_{cdl}, of the portion (P) of the artery lumen,
torsion, T_{cdl}, of the portion (P) of the artery lumen,
path length, PL_{cdl}, of the portion (P) of the artery lumen,
branching angle, BA_{cdl}, of the portion (P) of the artery lumen,
   wherein the portion (P) of the artery lumen has an end located at the candidate delivery location (130, a to c).

The set of vascular cost parameters preferably comprises at least:
the minimum diameter, D_{cdl},
the tortuosity, τ_{cdl}, and
the curvature, κ_{cdl}.

The method preferably further comprises:
determining, for each candidate delivery location (130, a to e), a (target) coverage, wherein:
the coverage is related to
   target coverage (TaC) that is related to a volume of target (120) contacted by the substance when delivered at the candidate delivery location (130, a to c) by a tip of the vascular catheter (200) compared with total volume of the target (120), and optionally
   non-target coverage (NTaC) that is related to a volume of non-target tissue within the organ contacted with the substance when delivered at the candidate delivery location (130, a to c) by the tip of the vascular catheter compared with total volume of the non-target tissue—
      wherein the evaluating comprises using a combination of the vascular cost and the coverage for each candidate delivery location (130, a to c).

The target coverage (TaC) is preferably determined by:
determining all downstream perfusion zones of the candidate delivery location (130, a to c), wherein each downstream perfusion zone is a three-dimensional region grown from a downstream outlet of the candidate delivery location (130, a to c), wherein the downstream outlet is an outlet of an artery lumen into the organ downstream of the candidate delivery location (130, a to c),
determining a target grown volume, V_{tg}, that is a summed total volume of downstream perfusion zones (134a₁ to a₅; 134b₁ to b₈; 134c₁ to c₄) for the candidate delivery location (130, a to c) disposed within the target (120),
determining a total target volume Vₜₜ, that is a total volume of the target, and
determining the target coverage comprising determining a ratio between V_{tg} and Vₜₜ.

The non-target coverage (NTaC) is preferably determined by:
determining all downstream perfusion zones of the candidate delivery location (130, a to c), wherein each downstream perfusion zone is a three-dimensional region grown from a downstream outlet of the candidate delivery location (130, a to c), wherein the downstream outlet is an outlet of an artery lumen into the organ downstream of the candidate delivery location (130, a to c)
determining, a non-target grown volume, V_{ntg}, that is a summed total volume of perfusion zones (134a₁ to a₅; 134b₁ to b₈; 134c₁ to c₄) for the candidate delivery location (130, a to c) disposed in non-target tissue within the organ,
determining a total non-target volume Vₜₙₜ, that is a total volume of non-target,
determining, the non-target coverage comprising determining a ratio between V_{ntg} and Vₜₙₜ.

The method preferably further comprises:
determining, for each candidate delivery location (130, a to c) a target dose uncertainty, wherein
   the target dose uncertainty is a measure of a likelihood that the substance flowing from the candidate delivery location (130, a to c) towards the target will flow through one or more branches leading into the target (200),
      wherein the evaluation comprises using a combination of the vascular cost and the target dose uncertainty for each candidate delivery location (130, a to c).

The target dose uncertainty for the candidate delivery location (130, a to c), is preferably determined comprising the steps:
obtaining a segment map (310), wherein the segment map (310) is:
   a transverse cross-section of an arterial lumen in the 3D model at the candidate delivery location (130, a to c);
   divided into a plurality of lumen segments (302, a to e);
predicting, for each candidate delivery location (130, a to c), a release of microparticles from the segment map (310) in a downstream direction, and a trajectory of each microparticle;
assigning to each lumen segment (302, a to e), a segment target dose fraction, STDF, value that is an indication of the quantity of microparticles released from that lumen segment (302, a to e) that flow into the target (120) compared with total quantity microparticles released from that lumen segment (302, m to p),
determining from the STDF values, a cluster target dose fraction, CTDF, wherein a cluster (306) is collection of lumen sections (302, b to e) grouped together to form a shape of a catheter tip (202) at a specific location on the segment map (310), and the CTDF is determined from the STDF values of lumen sections (302, b to e) within that cluster (306),
determining a plurality of the CTDF values for a sample group, wherein the each CTDF of the sample group is determined from a cluster having a different location on the segment map (310),
determining from the sample group of CTDF values an uncertainty range for the candidate delivery location (130, a to c), wherein the uncertainty range has one of more of
   a maximum CTDF value,
   a minimum CTDF value,
   a median CTDF value,
determining the target dose uncertainty from the uncertainty range.

The target dose uncertainty preferably comprises a tip uncertainty component, and wherein the tip uncertainty is
indicative of an uncertainty that the catheter tip (202) would be disposed in a cluster causing flow of substance into the target (120), and
determined comprising taking difference between the maximum CTDF value and the minimum CTDF value.

The target dose uncertainty preferably comprises an effective target dose component, and wherein a high effective target dose is
indicative of a proportion of the substance that is expected to reach the target (120), and
determined comprising taking the median CTDF value.

The method may further comprise:
determining, for each candidate delivery location (130, a to c) a radiation damage score, wherein
   the substance contains one or more radionuclides;
   the radiation damage score is a likelihood that the substance flowing from the candidate delivery location (130, a to c) towards the target will cause damage to tissue of the target (120);
      wherein the evaluation comprises using a combination of the vascular cost and the radiation damage score for each candidate delivery location (130, a to c).

The evaluation of a candidate delivery location (130, a to c) may comprise using a combination of the vascular cost, the coverage and the target dose uncertainty for the candidate delivery location (130, a to c).

The evaluation of a candidate delivery location (130, a to c) may comprise using a combination of the vascular cost, the coverage, the target dose uncertainty and the radiation damage score for the candidate delivery location (130, a to c).

The present method may be performed using a computer.

Further provided is a computing device or system configured for performing the method as described herein.

Further provided is a computer program or computer program product having instructions which when executed by a computing device or system cause the computing device or system to perform the method as described herein.

Further provided is a data stream which is representative of a computer program or computer program product having instructions which when executed by a computing device or system cause the computing device or system to perform the method as described herein.

### FIGURE LEGENDS

FIG. 1 is a 2D schematic representation of a 3D model of an organ, vasculature, and target.

FIG. 2 is the representation of FIG. 1, with three candidate delivery locations indicated.

FIG. 3 is the representation of FIG. 1, with one candidate delivery location serving multiple downstream outlets, and multiple perfusion zones (shown partially grown).

FIG. 4 is the representation of FIG. 1, with another candidate delivery location serving multiple downstream outlets, and multiple perfusion zones (shown partially grown).

FIG. 5 is the representation of FIG. 1, with another candidate delivery location serving multiple downstream outlets, and multiple perfusion zones (shown partially grown).

FIG. 6 is the representation of a transverse cross-section of an arterial lumen at a candidate delivery location showing a segment map and cluster.

FIGS. 7A to 7D segment maps of an arterial lumen at a candidate delivery location, wherein a cluster is indicated in different locations of the segment map (FIG. 7A: central; FIG. 7B lower right; FIG. 7C upper left; FIG. 7D lower left).

FIG. 8 is a 3D model of a tumour target and arteries feeding the tumour target.

FIG. 9 is a 3D model of a tumour target of FIG. 9, and three candidate delivery locations marked on the arteries feeding the tumour target and downstream healthy tissue.

FIG. 10 is a chart showing target dose fraction (TDF) for each candidate delivery location ([1], [2], [3]) for a sample group of 100 different lumen segments.

### DETAILED DESCRIPTION OF INVENTION

Before the present system and method of the invention are described, it is to be understood that this invention is not limited to the particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any 3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the present description of the invention, reference is made to the accompanying drawings that form a part hereof, and in which are shown by way of illustration only of specific embodiments in which the invention may be practiced. Parenthesized or emboldened reference numerals affixed to respective elements merely exemplify the elements by way of example, with which it is not intended to limit the respective elements. Unless otherwise indicated, all figures and drawings in this document are not to scale and are chosen for the purpose of illustrating different embodiments of the invention. In particular the dimensions of the various components are depicted in illustrative terms only, and no relationship between the dimensions of the various components should be inferred from the drawings, unless so indicated.

It is to be understood that other embodiments may be utilised and structural or logical changes may be made without departing from the scope of the present invention. The following detailed description, therefore, is not to be taken in a limiting sense, and the scope of the present invention is defined by the appended claims.

The term ‘downstream’ and ‘upstream’ are used herein to describe a relative location within an artery with respect to a reference point. The reference point is typically the location of the catheter tip. A location downstream of a reference point means the location receives blood flowing from the reference point, conversely a location upstream of a reference point means the reference point receives blood flowing from the location.

The term ‘transverse’ is used herein to mean in a direction perpendicular to a longitudinal. For instance, a transverse cross-section of a lumen is a section that is perpendicular to the lumen centerline.

The subject is the entity being evaluated. Typically the subject is mammalian, preferably human. The user is the person carrying out part or all of the evaluation. The user may be a qualified medical practitioner (e.g. interventional radiologist, surgeon), or a team thereof.

The target is tissue located within the organ. It is typically a tumour. Non-target tissue is tissue within the organ that is not a part of the target tissue.

The organ may be any bodily organ, in particular, liver, kidney, uterus, lungs, pancreas, prostate or brain.

The substance may contain an embolising agent. The embolising agent typically comprises a liquid suspension of microparticles. The microparticles in embolising agents are known in the art and typically have a diameter of 20 to 400 μm. When the procedure is TARE, the substance preferably contains a transarterial radioembolisation (TARE) agent. Examples of a TARE agents include ⁹⁰Y-loaded microspheres (e.g. SIR-Sphere® and TheraSphere®). When the procedure is transarterial chemoembolisation (TACE), the substance preferably contains a chemotherapeutic agent (e.g. microparticles loaded with chemotherapeutic agent).

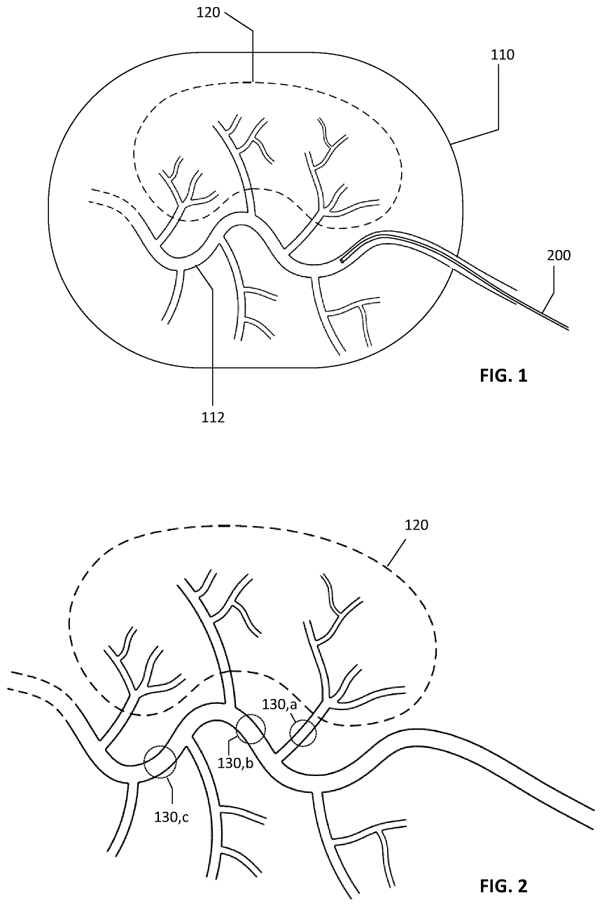

Provided herein is a method for evaluating a plurality of candidate delivery locations (130, a to c) for a substance by the vascular catheter (200) for treatment of a target (120) within an organ (110) of a subject. A schematic of an organ (110), vasculature lumens (112), and target (120) are shown in FIG. 1. Candidate delivery locations (130, a to c) are shown in FIG. 2.

The method comprises receiving a three-dimensional (3D) model of at least a part of the organ (110) of the subject. The method further comprises determining from the 3D model a vascular cost (VC) for each candidate delivery location (130, a to c) which is related to an accessibility of the candidate delivery location (130, a to c) by a tip of the vascular catheter. The evaluation comprises using the vascular cost for each candidate delivery location (130, a to c). In other words, each candidate delivery location (130, a to c) is evaluated, based on the vascular cost. The vascular cost (VC) is preferably a score value. Each parameter contributing to the VC may be a score value.

A score value is a scalar value on a scale having a lower limit and an upper limit (e.g. between 0 and 1, between 0 and 10, between 0 and 100). One end of the scale may be indicative of a highly suitable location for delivery of the substance by the vascular catheter (200) for treatment of the target (120) within the organ (110) of the subject. The other end of the scale may be indicative of an unsuitable location for delivery of the substance by the vascular catheter (200) for treatment of the target (120) within the organ (110) of the subject.

An upper limit to the scale may be indicative of a most suitable location, and a lower limit to the scale may be indicative of a most suitable location, or vice versa.

A value for the upper limit may be arbitrarily selected, as a guidance, it may be 1, 10, or 100. A value for the lower limit may be arbitrarily selected, as a guidance, it may be 0.

In order to generate a score value from a parameter (e.g. from vascular cost, or target coverage, and target dose uncertainty), the parameter is normalised such that it falls within the limits of the scale. The normalisation is typically performed comprising providing an extremity (most favoured or least favoured) value for the parameter, and creating a ratio between the measured parameter value and the extremity value parameter. A scale is created where a value of 1 represents one end of the scale, and ratios close to or equal to 1 are close to or equal to the extremity value. Depending on the ratio numerator and denominator the other end of the scale might be zero. The skilled person would appreciate that variations may be applied, for instance, to invert the scale (e.g. 0 most favourable and 1 least favourable, or vice versa) or to rescale the limits (multiply by 10, 100 or another value).

Where an evaluation is based on a combination of parameters (e.g. vascular cost and one or more of target coverage and target dose uncertainty) scalar values for each may be normalised to be on a separate scale, where each scale has the same range (e.g. 0 to 1), and the same extremes of the scale represent the most favourable or most unfavourable locations. Methods to normalise parameters, invert the scales or to rescale the limits are understood by the skilled person. The evaluation may then be based on multiple normalised scalar values e.g. average, weighted average.

The evaluation may be provided as one or multiple score values. The evaluation may be provided as a score value for vascular cost, and optionally target coverage (see elsewhere herein), and optionally target dose uncertainty (see elsewhere herein).

A three dimensional (3D) model is a model obtained from the 3D medical image. The 3D model comprises an indication of boundaries of the organ, target and arterial lumens, and their relative positions. The 3D model may be created using standard segmentation software such as Mimics (Materialise, Belgium), 3D Slicer (https://www.slicer.org/) or automatic segmentation tools. From the 3D model, the evaluation of the plurality of candidate delivery locations (130, a to c) may be made. The 3D medical image (also termed “medical image” herein) may be any 3D medical image obtained from the subject. The medical image is preferably obtained using a computed tomography (CT) scan or magnetic resonance imaging (MRI) scan of the subject. Ideally, medical image has a resolution (and slice thickness) that is at least a factor of 3 smaller than the minimal vessel diameters to segment.

Candidate delivery location (130, a to c) may be determined by the user (e.g. radiologist) or automatically. An automatic determination of the candidate delivery location (130, a to c) may be based on one or more of the following:
is within an arterial lumen within the organ; -is within an arterial lumen that feeds downstream the target, or is within an artery having one or more downstream branches that feeds the target;
is within an arterial lumen having a diameter above a certain threshold (e.g. larger than a catheter size);
   Preferably, the candidate delivery location (130, a to c) may be determined by excluding locations that do not feed downstream the target, and excluding locations that are below a certain threshold (e.g. smaller than a catheter size).

The vascular cost (VC_{cdl}) of a candidate delivery location (130, a to c) is a measure of difficulty in access by the catheter to the candidate delivery location (130, a to c).

The vascular cost (VC_{cdl}) of a candidate delivery location (130, a to c) is related to or determined from a set of VC parameters. The set of VC parameters comprises at least one, preferably all of the VC parameters of Table A.

TABLE A
VC parameters

| VC | | |
|---|---|---|
| parameter | Name | Description |
| D_{cdl} | Minimum Diameter | minimum diameter of arterial lumen upstream of the |
| | | candidate delivery location (130, a to c). Measured in |
| | | the same lumen as the candidate delivery location |
| | | (130, a to c). |
| τ_{cdl} | Tortuosity | tortuosity of arterial lumen upstream of the candidate |
| | | delivery location (130, a to c). Measured in the same |
| | | lumen as the candidate delivery location (130, a to c). |
| κ_{cdl} | Curvature | curvature of arterial lumen upstream of the candidate |
| | | delivery location (130, a to c) . Measured in the same |
| | | lumen as the candidate delivery location (130, a to c). |
| T_{cdl} | Torsion | torsion of arterial lumen upstream of the candidate |
| | | delivery location (130, a to c). Measured in the same |
| | | lumen as the candidate delivery location (130, a to c). |
| PL_{cdl} | Path length | path length of arterial lumen from the candidate |
| | | delivery location (130, a to c) to point the upstream of |
| | | the candidate delivery location (130, a to c). Measured |
| | | in the same lumen as the candidate delivery location |
| | | (130, a to c). |
| BA_{cdl} | Branching angle | Branching angle of arterial lumen upstream of the |
| | | candidate delivery location (130, a to c) . Measured in |
| | | the same lumen as the candidate delivery location |
| | | (130, a to c). |

Each of the VC parameters is determined for a portion (P) of the artery lumen length. A first (terminal) end of the portion is located at the candidate delivery location (130, a to c), and a second (terminal) end of the portion is located upstream of first (terminal) end. The second (terminal) end of the portion is preferably located at the arterial entry to the organ. The portion may include the arterial entry to the organ. The portion may extend upstream of the arterial entry to the organ. The portion is a portion of the artery lumen to be taken by the catheter to reach the candidate delivery location (130, a to c).

The value of the minimum diameter (D_{cdl}) of a candidate delivery location is determined as the smallest arterial lumen diameter upstream of the candidate delivery location (130, a to c) disposed within said arterial lumen. The diameter of an arterial lumen at a location under consideration is a diameter of the largest circle that will fit within the transverse cross-section of the arterial lumen at the location under consideration. Alternatively, the diameter of an arterial lumen at a location under consideration may be a diameter of a circle that has an area equal to the transverse cross-sectional area of the arterial lumen at the location under consideration. The value of the minimum diameter (D_{cdl}) may be determined for the portion (P) of the artery lumen. The smallest diameter within the portion (P) is the value of the minimum diameter (D_{cdl}).

The minimum diameter may be normalised to the smallest value obtained for all candidate delivery locations (see Eq. 1), viz:

$${\overline{D}}_{cdl} = \frac{min {D}_{c d l s}}{{D}_{c d l}} \qquad [ Eq . 1 ]$$

where:
D̅_{cdl} is the normalised minimum diameter for the candidate delivery location under consideration,
D_{cdl} is the minimum diameter of the candidate delivery location under consideration,
minD_{cdls} is the smallest value of the minimum diameters measured for all the candidate delivery locations under consideration (extremity value).

A D̅_{cdl} value of 1 represents one end of the scale, and a D̅_{cdl} close to or equal to 1 is close to or equal to a most favourable value. A D̅_{cdl} value close to 0 is close to a most favourable value. The greater the value of D̅_{cdl} on the scale between 0 and 1, the less favourable the candidate delivery location.

Value of the tortuosity (τ_{cdl}) of a candidate delivery location (130, a to c) is determined as a deviation, from a straight line, of a path followed by the portion (P) of the artery lumen. Tortuosity of a path is known in the art. The path is preferably a centre line of the lumen of the portion (P) of the artery.

The tortuosity (τ_{cdl}) of the candidate delivery location (130, a to c) is determined from a ratio between a length (Lₗᵤₘₑₙₚₐₜₕ) of the lumen path between first end and second end of the portion (P), and a length (L_{straightpath}) of a straight (rectilinear) path between first end and second end of the portion (P). The tortuosity (τ_{cdl}) may be such that a value of zero represents no deviation from the straight path, and a value greater than zero represents increasing deviation from the straight path.

The tortuosity (τ_{cdl}) may be calculated from the formula (Eq. 2):

$${τ}_{cdl} = \frac{{L}_{l u m e n p a t h}}{{L}_{straightpath}} - 1 \qquad [ Eq . 2 ]$$

where
τ_{cdl} is the tortuosity (τ_{cdl}) of the candidate delivery location
Lₗᵤₘₑₙₚₐₜₕ is a length of the lumen path between first end and second end of the portion (P), and L_{straightpath} is a length of a straight (rectilinear) path between first end and second end of the portion (P).

The greater the value of τ_{cdl} the less favourable the candidate delivery location.

The centre line of the lumen of the artery may be generated using any suitable method, for instance, using the software Mimics (Materialise, Belgium).

The tortuosity may be normalised to the largest value obtained for all candidate delivery locations (see Eq. 3), viz:

$${\overline{τ}}_{cdl} = \frac{{τ}_{c d l}}{max {τ}_{c d l s}} \qquad [ Eq . 3 ]$$

where:
   τ̅_{cdl} is the normalised tortuosity for the candidate delivery location under consideration,
   τ_{cdl} is the tortuosity of the candidate delivery location under consideration,
   maxτ_{cdls} is the largest value of tortuosity measured for all the candidate delivery locations under consideration (extremity value).

A τ̅_{cdl} value of 1 represents one end of the scale, and a τ̅_{cdl} close to or equal to 1 is close to or equal to a most unfavourable value. A τ̅_{cdl} value close to 0 is close to a most favourable value. The greater the value of τ̅_{cdl} on the scale between 0 and 1, the less favourable the candidate delivery location.

Value of the curvature (κ_{cdl}) of a candidate delivery location (130, a to c) is determined as a curvature of the path followed by the portion (P) of the artery lumen. Curvature of a path is known in the art. The path is preferably a centre line of the lumen of the portion (P) of the artery. The centre line of the lumen of the artery may be generated using any suitable method, for instance, using the software Mimics (Materialise, Belgium).

Value of the curvature (κ_{cdl}) of a candidate delivery location is preferably determined from first and second derivatives of the path. The curvature (κ_{cdl}) may be calculated from the formula (Eq. 4):

$${κ}_{cdl} = \frac{‖ \dot{c} \times \ddot{c} ‖}{{\vert \dot{c} \vert}^{3}} \qquad [ Eq . 4 ]$$

where:
κ_{cdl} is curvature of the candidate delivery location
ċ is the first derivative of the lumen path between first end and second end of the portion (P);
\ddot{c} is the second derivative of the lumen path between first end and second end of the portion (P);
x is a cross product between two vectors.

The first and second derivatives may be approximated as central finite difference.

The greater the curvature, the greater the value of κ_{cdl}. The greater the value of κ_{cdl} the less favourable the candidate delivery location.

The curvature may be normalised to the largest value obtained for all candidate delivery locations (see Eq. 5), viz:

$${\overline{κ}}_{cdl} = \frac{{κ}_{c d l}}{max {κ}_{c d l s}} \qquad [ Eq . 5 ]$$

where:
κ̅_{cdl} is the normalised curvature for the candidate delivery location under consideration,
κ_{cdl} is the curvature of the candidate delivery location under consideration,
maxκ_{cdls} is the largest value of curvature measured for all the candidate delivery locations under consideration (extremity value).

A κ̅_{cdl} value of 1 represents one end of the scale, and a κ̅_{cdl} close to or equal to 1 is close to or equal to a most unfavourable value. A κ̅_{cdl} value close to 0 is close to a most favourable value. The greater the value of κ̅_{cdl} on the scale between 0 and 1, the less favourable the candidate delivery location.

Value of the torsion (T_{cdl}) of a candidate delivery location (130, a to c) is determined as a torsion of the path followed by the portion (P) of the artery lumen along its length. Measurement of torsion has been described, for instance, in Piccinelli et al, IEEE Trans Med Imaging. 2009 August; 28(8):1141-55. doi: 10.1109/TMI.2009.2021652. The path is preferably a centre line of the lumen of the portion (P) of the artery. Torsion defines how much the path twists in space. The path is preferably a centre line of the lumen of the portion (P) of the artery. The centre line of the lumen of the artery may be generated using any suitable method, for instance, using the software Mimics (Materialise, Belgium).

The torsion (T_{cdl}) may be calculated from the formula (Eq. 6):

$${T}_{cdl} = \frac{( \dot{c} \times \ddot{c} ) \cdot {c}^{…}}{{‖ \dot{c} \times \ddot{c} ‖}^{2}} \qquad [ Eq . 6 ]$$

where:
T_{cdl} is torsion of the candidate delivery location;
ċ is the first derivative of the lumen path between first end and second end of the portion (P);
\ddot{c} is the second derivative of the lumen path between first end and second end of the portion (P).
[symbol] is the third derivative of the lumen path between first end and second end of the portion (P).

The first and second derivatives may be approximated as central finite difference.

The torsion may be normalised to the largest value obtained for all candidate delivery locations (see Eq. 7), viz:

$${\overline{T}}_{cdl} = \frac{{T}_{c d l}}{max {T}_{c d l}} \qquad [ Eq . 7 ]$$

where:
   T̅_{cdl} is the normalised torsion of the candidate delivery location under consideration;
   T_{cdl} is the torsion of the candidate delivery location under consideration;
   maxT_{cdl} is the largest value of torsion measured for all the candidate delivery locations under consideration (extremity value).

A T̅_{cdl} value of 1 represents one end of the scale, and a T̅_{cdl} close to or equal to 1 is close to or equal to a most unfavourable value. A T̅_{cdl} value close to 0 is close to a most favourable value. The greater the value of T̅_{cdl} on the scale between 0 and 1, the less favourable the candidate delivery location.

The value of the path length (PL_{cdl}) of a candidate delivery location (130, a to c) is determined as the length of the lumen path between the first end and second end of the portion (P). The path is preferably a centre line of the lumen of the portion (P) of the artery. The centre line of the lumen of the artery may be generated using any suitable method, for instance, using the software Mimics (Materialise, Belgium).

The path length (PL_{cdl}) is equal to the lumen path length (Lₗᵤₘₑₙₚₐₜₕ) mentioned above.

The path length (PL_{cdl} may be normalised to the largest value obtained for all candidate delivery locations (see Eq. 8), viz:

$${\overline{PL}}_{cdl} = \frac{P {L}_{c d l}}{max P {L}_{c d l}} \qquad [ Eq . 8 ]$$

where:
   P̅L̅_{cdl} is the normalised path for the candidate delivery location under consideration;
   PL_{cdl} is the path length of the candidate delivery location under consideration;
   maxPL_{cdl} is the largest value of torsion measured for all the candidate delivery locations under consideration (extremity value).

A P̅L̅_{cdl} value of 1 represents one end of the scale, and a P̅L̅_{cd} close to or equal to 1 is close to or equal to a most unfavourable value. The greater the value of PL_{cdl} the less favourable the candidate delivery location.

A P̅L̅_{cdl} value of 1 represents one end of the scale, and a P̅L̅_{cdl} close to or equal to 1 is close to or equal to a most unfavourable value. A P̅L̅_{cdl} value close to 0 is close to a most favourable value. The greater the value of P̅L̅_{cdl} on the scale between 0 and 1, the less favourable the candidate delivery location.

Value of the branching angle (BA_{cdl}) of a candidate delivery location (130, a to c) is a measure of a change in path angle caused by the presence of a branching point in the portion (P) of the artery lumen. When the path, in an upstream-downstream direction in the portion (P) of the artery lumen, encounters a branching point, it changes direction as it passes into one of the branches downstream of the branching point. The branching angle (BA_{cdl}) is a measure of an angle between the path (upstream portion) before it enters the branching point and the path (downstream portion) after it leaves the branching point and occupies only one of the branches. The branching point may be a point of bifurcation, trifurcation, or other order of splitting Branching angle of a path is known in the art. The path is preferably a centre line of the lumen of the portion (P) of the artery. The centre line of the lumen of the artery may be generated using any suitable method, for instance, using the software Mimics (Materialise, Belgium).

Value of the branching angle (BA_{cdl}) is preferably determined by assigning three points (A, B, C) along the path followed by the portion (P) of the artery lumen, wherein point B is at the branching point, point A is upstream of the branching point, and point C is downstream of the branching point and within one of the branches. Value of the branching angle (BA_{cdl}) is the absolute value of (180−angle (D)), where angle (D) is an angle between the straight line between B-C and straight line between B-A. The straight line between B-C and/or the straight line between B-A may be extrapolated so that they cross at B. The linear distance between B-C and B-A is the same. The linear distance between B-C and B-A is preferably between 1 and 2 mm.

Where the portion (P) of the artery lumen contains a plurality of consecutive branches, the branching angle (BA_{cdl}) used to determine the vascular cost (VC_{cdl}) is the largest branching angle (BA_{cdl}) within the portion (P).

The branching angle (BA_{cdl}) in degrees is a value in a range >0 deg to <180 deg, more typically >0 deg to <180 deg. The greater the branching angle, the greater the value of BA_{cdl}. The greater the value of BA_{cdl} the less favourable the candidate delivery location.

The branching angle (BA_{cdl}) may be normalised to the largest value (180) obtainable for a candidate delivery location (see Eq. 9), viz:

$${\overline{BA}}_{cdl} = \frac{B {A}_{cdl}}{180} \qquad [ Eq . 9 ]$$

where:
   B̅A̅_{cdl} is the normalised branching angle for the candidate delivery location under consideration,
   BA_{cdl} is the branching angle of the candidate delivery location under consideration,
   180 is the largest value of branching angle obtainable

A B̅A̅_{cdl} value of 1 represents one end of the scale, and a B̅A̅_{cdl} close to or equal to 1 is close to or equal to a most unfavourable value. A B̅A̅_{cdl} value close to 0 is close to a most favourable value. The greater the value of B̅A̅_{cdl} on the scale between 0 and 1, the less favourable the candidate delivery location.

The vascular cost (VC_{cdl}) for a candidate delivery location may be a score value. The vascular cost (VC_{cdl}) for a candidate delivery location may be a score value on a scale having a lower limit and an upper limit (e.g. between 0 and 1, between 0 and 10, between 0 and 100). One end of the scale (e.g. the upper limit (1, 10, 100) may be indicative of a high difficulty (high cost) in access by the vascular catheter (200) for treatment of the target (120) within the organ (110) of the subject. The other end of the scale (e.g. the lower limit (0)) may be indicative of a low difficulty (low cost) in access by the vascular catheter (200) for treatment of the target (120) within the organ (110) of the subject.

The vascular cost (VC_{cdl}) for a candidate delivery location may be determined as a scalar (score) value that is an average of one or multiple of the normalised VC parameters. For example, the vascular cost (VC_{cdl}) for a set of VC parameters may be determined as follows when using the average values of the VC parameters (see Eq. 10):

$$V {C}_{cdl} = \frac{\sum \overline{VC} {parameter}_{n}}{n} \qquad [ Eq . 10 ]$$

where
   ΣV̅C̅parameterₙ is the sum of the n normalised VC parameters in the set
n is the number of normalised VC parameters in the set.

Alternatively or in addition, the vascular cost (VC_{cdl}) for a candidate delivery location may be determined as a scalar (score) value that is an average of weighted, normalised VC parameters in the set. For example, the vascular cost (VC_{cdl}) may be determined as (Eq. 11):

$$V {C}_{cdl} = \frac{\sum {c}_{n} \overline{VC} {parameter}_{n}}{n} \qquad [ Eq . 11 ]$$

wherein
   ΣcₙV̅C̅parameterₙ is the sum of the n normalised VC parameters in the set, each parameter multiplied by a weight cₙ.
n is the number of normalised VC parameters

One or more of the weights may be set to one (i.e. no weighting effect) or zero (i.e. neglecting a parameter). A default weight may be one. One or more of the weights may be set to a value other than one. For instance, the user (e.g. practitioner) may be highly skilled and might give a low weight to τ̅_{cdl} in order to minimise impact of a high scoring tortuosity on VC.

The evaluation may be provided as one or multiple score values. The evaluation may be provided as a score value for vascular cost.

Alternatively or in addition, the vascular cost (VC_{cdl}) for a candidate delivery location may be a list of individual VC parameters, expressed as non-normalised or normalised values.

The method may further comprise determining from the 3D model a coverage (C) for each candidate delivery location (130, a to c) which is related to
a volume of target (120) contacted with the substance when delivered at the candidate delivery location (130, a to c) by the tip of the vascular catheter (known as target coverage (TaC) herein), and optionally
a volume of non-target contacted with the substance when delivered at the candidate delivery location (130, a to c) by the tip of the vascular catheter (known as non-target coverage (NTaC) herein).

The target coverage (TaC) is related to a volume of target tissue contacted by the substance delivered at the candidate delivery location (130, a to c) compared with total volume of the target. Ideally, all the substance flows from the candidate delivery location (130, a to c) to the target.

The non-target (NTaC) coverage is related to a volume of non-target tissue contacted by the substance delivered at the candidate delivery location (130, a to c) compared with total volume of the non-target. Ideally, none the substance flows from the candidate delivery location (130, a to c) to the non-target. The non-target coverage can depend on the total volume of the non-target.

The coverage (TaC and optionally NTaC) is determined by identifying outlets (132) in the 3D model, and using each outlet as a seedpoint for growing a region, known as a perfusion zone (134), therefrom. An outlet (132, a_{1 to 5}; 132, b_{1 to 13}; 132, c_{1 to 5}) is an outlet of an artery having a lumen in fluid with the organ (target or non-target tissue), wherein the outlet is also present within the organ. The outlet is identified from the 3D model based on a location where the artery lumen terminates within the organ and/or target.

Perfusion zones (134) grown from outlets (132) downstream of the candidate delivery location (130, a to c) are used to calculate the coverage for the candidate delivery location (130, a to c). By an outlet being downstream of the candidate delivery location, it is meant that the outlet is located such that blood flows from the candidate delivery location towards the outlet. In particular, the size (e.g. volume) of perfusion zones downstream of the candidate delivery location (130, a to c) are used to calculate the coverage for the candidate delivery location (130, a to c).

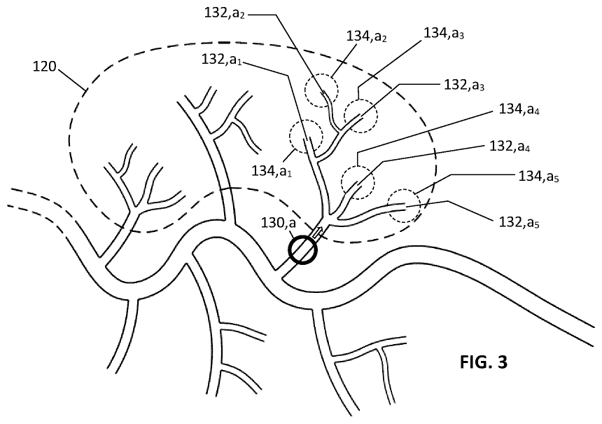

Exemplarily, FIG. 3 highlights multiple downstream outlets (132, a_{1 to 5}) for one of the candidate delivery locations (130, a), and multiple perfusion zones (134, a_{1 to 5}) (shown partially grown). In the figure, one perfusion zone (134, a_{1 to 5}) (shown partially grown) for each and every downstream outlet (132, a_{1 to 5}) of the candidate delivery location (130, a) is illustrated, making a total of 5 perfusion zones. All the outlets (132, a_{1 to 5}) downstream of the delivery location are located in the target (120).

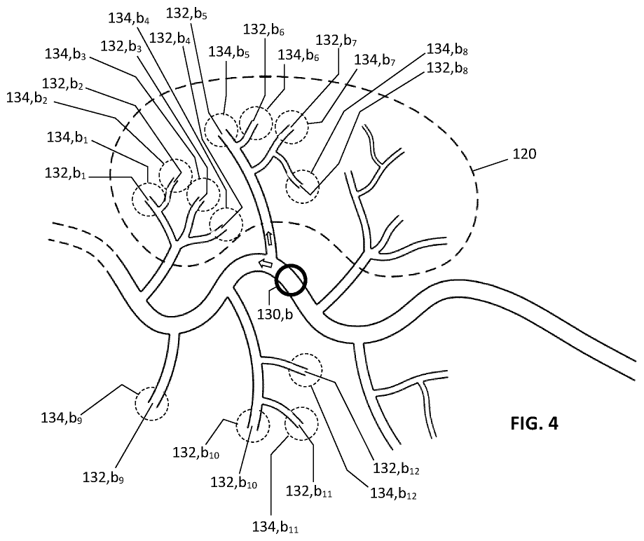

Exemplarily, FIG. 4 shows multiple downstream outlets (132, b_{1 to 13}) for another candidate delivery location (130, b), and multiple perfusion zones (134, b_{1 to 13}) (shown partially grown), one perfusion zone (134, b_{1 to 13}) for each downstream outlet (132, b_{1 to 13}) of the candidate delivery location (130, b). Some outlets (132, b_{1 to 8}) downstream of the delivery location are located in the target (120); some outlets (132, b_{9 to 12}) downstream of the delivery location are located in non-target (e.g. healthy) tissue (120).

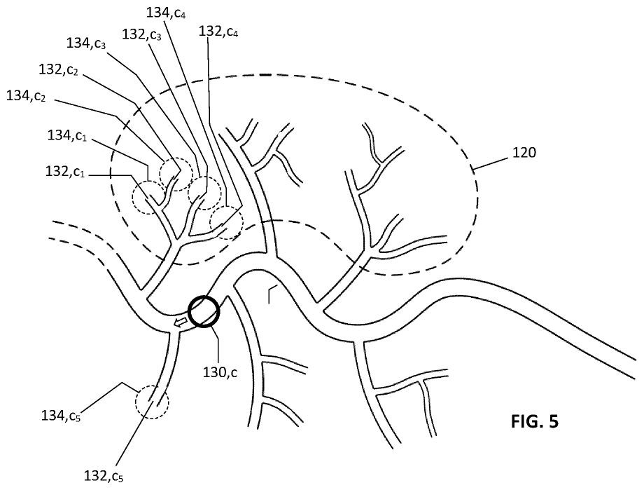

Exemplarily, FIG. 5 shows multiple downstream outlets (132, c_{1 to 5}) for another candidate delivery location (130, c), and multiple perfusion zones (134, c_{1 to 5}) (shown partially grown), one perfusion zone (134, c_{1 to 5}) for each downstream outlet (132, c_{1 to 5}) of the candidate delivery location (130, c). Some outlets (132, c_{1 to 4}) downstream of the delivery location are located in the target (120); one outlet (132, c₅) downstream of the delivery location is located in non-target (e.g. healthy) tissue (120).

Each perfusion zone (134, a_{1 to 5}; 134, b_{1 to 12}; 134, c_{1 to 5}) is a three dimensional region grown using a region growing protocol. Region growing protocols are known in the art (e.g. Vermijs, Saar, et al. OncoDot.3 Symposium, Abstracts, 2021; Pratondo, A. et al (2014) “Region Growing for Medical Image Segmentation Using a Modified Multiple-seed Approach on a Multi-core CPU Computer” In: Goh, J. (eds) The 15th International Conference on Biomedical Engineering. IFMBE Proceedings, vol 43. Springer, Cham). Each perfusion zone is grown simultaneously. Each perfusion zone is grown at the same rate. Each perfusion zone is grown in three-dimensions. Growth of a perfusion zone is stopped when perfusion zone touches a boundary. A boundary may be an edge of the target (e.g. tumour), an edge of vasculature, edge of the organ, and a perfusion zone from another outlet.

A target grown volume (V_{tg}) is determined, that is a summed total volume of perfusion zones (for a candidate delivery location) disposed within the target. Exemplarily, in FIG. 3 the target grown volume for one of the candidate delivery locations (130, a) is the summed individual perfusion zone volumes located within the target (120), namely (vol (134, a₁)+vol (134, a₂)+vol (134, a₃)+vol (134, a₄)+vol (134, a₅)).

Exemplarily, in FIG. 4 the target grown volume for another candidate delivery location (130, b) is the summed individual perfusion zone volumes located within the target (120), namely: (vol (134, b₁)+vol (134, b₂)+vol (134, b₃)+vol (134, b₄)+vol (134, b₅)+vol (134, b₆)+vol (134, b₇)+vol (134, b₅)).

Exemplarily, in FIG. 5 the target grown volume for one of the candidate delivery locations (130, c) is the summed individual perfusion zone volumes located within the target (120), namely: (vol (134, c₁)+vol (134, c₂)+vol (134, c₃)+vol (134, c₄)).

A total target volume (Vₜₜ) may be determined, that is a total volume of the target.

A total non-target volume (Vₜₙₜ) may be determined, that is a total volume of the non-target tissue. Total non-target volume (Vₜₙₜ) may be calculated from total volume of the organ minus total volume of total target volume (Vₜₜ).

A non-target grown volume (V_{ntg}) may be determined that is a total volume of perfusion zones (for a candidate delivery location (130, a)) not disposed within the target.

Exemplarily, in FIG. 3 the non-target grown volume for one of the candidate delivery locations (130, a) is the summed individual perfusion zone volumes located within the non-target, namely zero.

Exemplarily, in FIG. 4 the non-target grown volume for another candidate delivery location (130, b) is the summed individual perfusion zone volumes located within the non-target, namely: (vol (134, b₉)+vol (134, b₁₀)+vol (134, b₁₁)+vol (134, b₁₂)).

Exemplarily, in FIG. 5 the non-target grown volume for one of the candidate delivery locations (130, c) is the summed individual perfusion zone volumes located within the non-target, namely: (vol (134, c₅)).

The target coverage (TaC) may be determined comprising determining a ratio between V_{tg} and Vₜₜ. A non-target coverage (NTaC) may optionally be determined comprising determining a ratio between V_{ntg} and Vₜₙₜ.

The target coverage (TaC) for a candidate delivery location may be a scalar (score) value. The target coverage for a candidate delivery location may be a scalar (score) value on a scale having a lower limit and an upper limit (e.g. between 0 and 1, between 0 and 10, between 0 and 100). One end of the scale (e.g. the upper limit (1, 10, 100) may be indicative of a high coverage of the target by the substance released at the candidate delivery location. The other end of the scale (e.g. the lower limit (0)) may be indicative of a low coverage of the target by the substance released at the candidate delivery location.

The target coverage may be a scalar (score) value related to the target grown volume compared with total volume of the target. For instance, target coverage may be based on the equation (Eq. 12):

$$T a C = \frac{{V}_{tg}}{{V}_{t t}} - 1 \qquad [ Eq . 12 ]$$

where:
TaC is target coverage for a candidate delivery location;
V_{tg} is target grown volume for the candidate delivery location;
Vₜₜ is total target volume (extremity value).

The value of TaC is already normalised. A TaC value of 1 represents one end of the scale, and a TaC close to or equal to 1 is close to or equal to a most unfavourable value. A TaC value close to 0 is close to a most favourable value. The lower the value of TaC on the scale between 0 and 1, the more favourable the candidate delivery location.

The non-target coverage (NTaC) for a candidate delivery location may be a scalar (score) value. The non-target coverage for a candidate delivery location may be a scalar (score) value on a scale having a lower limit and an upper limit (e.g. between 0 and 1, between 0 and 10, between 0 and 100). One end of the scale (e.g. the upper limit (1, 10, 100) may be indicative of a high coverage of the target by the substance released at the candidate delivery location. The other end of the scale (e.g. the lower limit (0)) may be indicative of a low coverage of the target by the substance released at the candidate delivery location.

The non-target coverage may be a scalar (score) value related to the non-target grown volume compared with the total volume of non-target. For instance, non-target coverage may be based on the equation (Eq. 13):

$$N T a C = \frac{{V}_{n t q}}{{V}_{t n t}} \qquad [ Eq . 13 ]$$

where:
NTaC is non-target coverage for a candidate delivery location;
V_{ntg} is non-target grown volume for the candidate delivery location;
Vₜₙₜ is total volume of non-target

The value of NTaC is already normalised. A NTaC value of 1 represents one end of the scale, and a NTaC close to or equal to 1 is close to or equal to a most unfavourable value. A NTaC value close to 0 is close to a most favourable value. The lower the value of NTaC on the scale between 0 and 1, the more favourable the candidate delivery location.

The coverage (C) for a candidate delivery location may be a scalar (score) value. The coverage for a candidate delivery location may be a scalar (score) value on a scale having a lower limit and an upper limit (e.g. between 0 and 1, between 0 and 10, between 0 and 100). One end of the scale (e.g. the upper limit (1, 10, 100) may be indicative of a high coverage of the target by the substance released at the candidate delivery location. The other end of the scale (e.g. the lower limit (0)) may be indicative of a low coverage of the target by the substance released at the candidate delivery location.

A coverage (C) value of 1 represents one end of the scale, and a coverage (C) close to or equal to 1 is close to or equal to a most unfavourable value. A coverage (C) value close to 0 is close to a most favourable value. The lower the value of coverage (C) on the scale between 0 and 1, the more favourable the candidate delivery location.

Coverage (C) is equal to the target coverage (TaC), or non-target coverage (NTaC), or to a combination of TaC and NTaC. The combination of TaC and NTaC is preferably an average of TaC and NTaC or a weighted average of TaC and NTaC.

The evaluating comprises using the vascular cost and coverage, for each candidate delivery location (130, a to c). In other words, each candidate delivery location (130, a to c) is evaluated, based on a combination of the vascular cost and coverage.

The evaluation (based on vascular cost and coverage) may be provided as one or more score values. The evaluation may be provided as a score value for vascular cost and a separate score value for coverage. The evaluation may be provided as a combined score value for vascular cost and coverage. The combined score is preferably an average of individual scores or an average of weighted individual scores for vascular cost and coverage.

The method may further comprise determining from the 3D model a target dose uncertainty for each candidate delivery location (130, a to c) which is related to a likelihood that the substance flowing from the candidate delivery location (130, a to c) towards the target will flow through one or more arterial branches converging with the target, and into the target when delivered at the candidate delivery location (130, a to c) by the tip of the vascular catheter. As described later below, the target dose uncertainty has one or two or three components: ‘tip uncertainty’ and/or ‘effective target dose’ and/or ‘uncertainty range’.

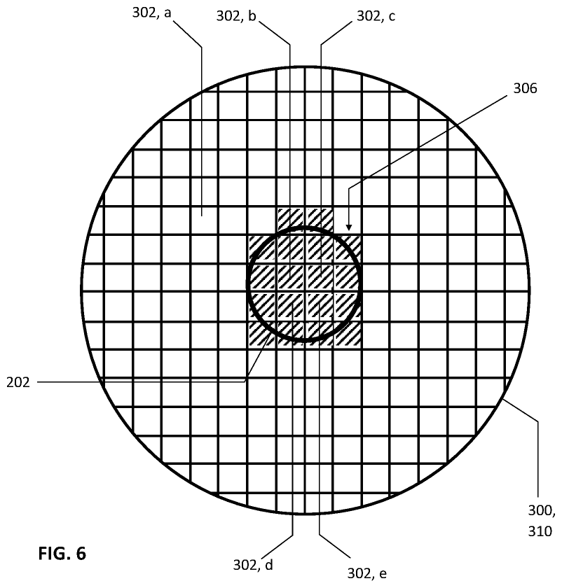

In order to determine target dose uncertainty, a transverse cross-section is taken of a lumen in the 3D model at each of the candidate delivery locations. The transverse cross-section (300) of the lumen at each candidate delivery location (130, a to c) is divided into a plurality of lumen segments (302, a to e). The divided transverse cross-section is known as a “segment map (310)”. An exemplary transverse cross-section (300) and segment map (310) is shown in FIG. 6. A lumen segment (302, a to e) is a region or box of a grid (FIG. 6). Each lumen segment has a different (x-y) location on the segment map (310). Different lumen segments may not overlap. The quantity of lumen segments (302, a to e) in the segment map (310) may depend on the size of transverse cross-section of the lumen. As a general guidance, square segments having a side edge length of 0.05 to 0.15 mm may be employed as lumen segments.

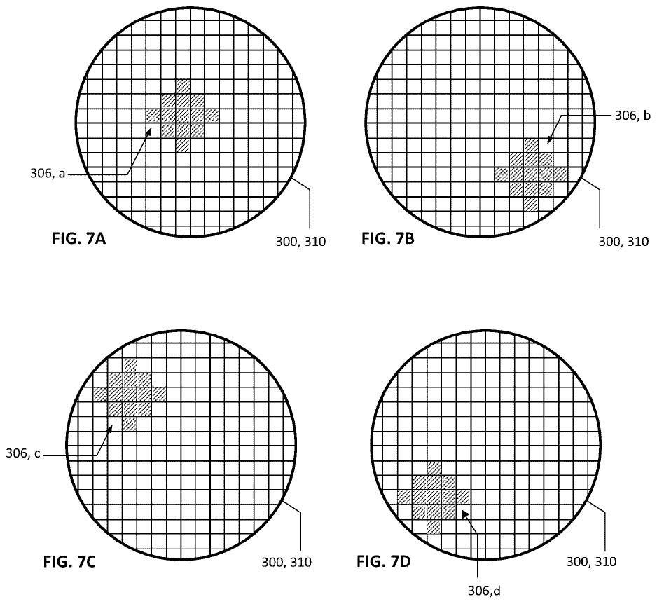

The catheter tip (202) is represented as a cluster (306) of lumen segments (302) on the segment map (310). A cluster (306) is a (x-y) location-specific collection of lumen segments (302, b to e) clustered (grouped) together to form a shape of a catheter tip (202). The lumen segments (302, b to e) within a cluster (306) are mutually adjacent. A cluster (306) may be positioned at any location on segment map (310). The size and shape of a cluster reflects the size and shape of the catheter tip. An exemplary cluster (306) and corresponding catheter tip (202) are shown in FIG. 6. FIGS. 7A to 7D show clusters (306, a to d) of the same size, each located at a different locations on the segment map (310). The quantity of lumen segments in a cluster may be dependent upon the area of the opening of the catheter tip. A smaller opening may be reflected by a lower quantity of lumen segments compared with a larger opening. In particular, a smaller opening may be reflected by a lower quantity of lumen segments distributed around a central segment of the transverse cross-section in the sample group compared with a larger opening.

The (x,y) location of the cluster (306, a to d) on the segment map can lead to a different outcome in terms of distribution of the substance. A cluster (306, a) located for instance towards the centre of the segment map (310) (e.g. FIG. 7A) may lead to increased perfusion and targeting of the target compared with cluster (306, d) located example towards the lower left of the segment map (310) (e.g. FIG. 7D)

The user can mainly control the axial location of the catheter tip within the lumen. However, the user cannot adjust position of the tip at the candidate delivery location in the transverse-cross sectional plane. Hence, the user is unable to steer the tip into a more favourable lumen segment(s). Furthermore, the user typically does not have knowledge of the lumen segment(s) where the tip is disposed at the candidate delivery location.

The target dose uncertainty is related to a probability that the cluster (306) (equating to catheter tip), disposed at a random location on the segment map (310) (equating to an (x-y) cross-sectional location of the lumen at one candidate delivery location (130, a to c)) will perfuse substance to the target, and to a probable dose that is effectively received by the target at the random location. As described later below, the target dose uncertainty has one or two components: ‘tip uncertainty’ and/or ‘effective target dose’.

Determining target dose uncertainty comprises computational fluid dynamics (CFD) applied to the 3D model to predict flow of substance from a candidate delivery location (130, a to c). This is preferably implemented using a simulation to predict flow of microparticles starting from an injection plane that is the segment map (310), wherein the flow proceeds in a downstream direction. For a selected lumen segment (302, a to e) of the segment map, a set of microparticles is released in simulation. The lumen segment microparticles are initially spaced (e.g. randomly) across an area of the lumen segment (302). All the microparticles of the set are released from the lumen segment simultaneously. The quantity of microparticles per lumen segment may be a value in the range 2 to 100, preferably 2 to 50. The size of a microparticle is preferably a value in the range 15 to 300 μm, preferably 20 to 100 μm. Each microparticle is preferably spherical.

In simulation, for the lumen segment (306), the downstream trajectory each microparticle of the set of microparticles released starting from that lumen segment is predicted. An example of a protocol for predicting flow of a set of microparticles is disclosed, for instance, in Bomberna et al., Frontiers in Bioengineering and Biotechnology, 2022. The proportion of microparticles released in a downstream direction starting from that lumen segment that is predicted to flow into one or more outlets (e.g. of a target or non-target, preferably target) is determined.

A segment target dose fraction (STDF) may be obtained for the set of microparticles of the (one) lumen segment (306, a to e). A segment target dose fraction (STDF) may be assigned to each lumen segment (302, a to e) that has a value indicative of a quantity of microparticles released from that lumen segment (302, a to e) that flowed into the target (120) compared with total quantity of microparticles released from that lumen segment (302, a to e). Calculation of the STDF of a segment preferably comprises (quantity of microparticles released from that lumen segment (302, a to e) that flowed into the target (120))/(total quantity of microparticles released from that lumen segment (302, a to e)).

If all microparticles released from a lumen segment (302, a to e) exit through one or more outlets flowing into the target (120), that lumen segment (e.g. 302, a to e) may be assigned an STDF value indicative of a high association between the lumen segment and target (e.g. 1). If microparticles released from a lumen segment exit through multiple outlets, some flowing into the target and some flowing into non-target tissue, an STDF value indicative of a lower association between the lumen segment and target may be assigned to the lumen segment (e.g. <1).

By calculating multiple STDF values for lumen segments (302, b to e) within a cluster (306), a cluster target dose fraction (CTDF) for that cluster is determined. The CTDF is determined by combining the multiple STDFs of the cluster. The combination is representative of the total number of microparticles in the cluster (306) that flowed into the target (120)) compared with total quantity of microparticles released from the cluster. Calculation of the CTDF of a cluster preferably comprises (quantity of microparticles released from that cluster (306) that flowed into the target (120))/(total quantity of microparticles released from that cluster).

To determine a cluster target dose fraction (CTDF) for a cluster, multiple sets of microparticles are released simultaneously starting from multiple lumen segments, giving rise to multiple STDFs.

The multiple lumen segments (302)/multiple STDFs are preferably all the lumen segments (302, a to e) of the segment map (310), and a selection of lumen segments (302, b to e) corresponding to a cluster (306) is later made. Determining multiple STDFs for all the lumen segments of the segment map (310) allows an efficient single simulation, and a later selection of a plurality of different locations of the cluster (306)/catheter tip (202) from the segment map (310) without necessarily having to perform further simulations. It is understood that additional simulations may be performed to improve accuracy. Thereby, a sample group containing a plurality of clusters and a plurality of cluster target dose fraction (CTDF) values (see below) can be determined from a minimum of one simulation.

Alternatively, multiple lumen segments (302)/multiple STDFs may be limited to those lumen segments (302, b to e) within a cluster. Multiple sets of microparticles may be released simultaneously from multiple lumen segments of a cluster at a first location in a first simulation, and for other locations, multiple sets of microparticles may be released therefrom in one or more further simulations. Thereby, a sample group containing a plurality of clusters and a plurality of cluster target dose fraction (CTDF) values (see below) is formed through multiple simulations.

If all microparticles released from a cluster (302, b to e) exit through one or more outlets flowing into the target (120), that cluster (e.g. 302, b to e) may be assigned a CTDF value indicative of a high association between the lumen segment and target (e.g. 1). If microparticles released from a cluster exit through multiple outlets, some flowing into the target and some flowing into non-target tissue, a CTDF value indicative of a lower association between the lumen segment and target may be assigned to the lumen segment (e.g. <1).

For each candidate delivery location, a sample group may be formed of a plurality of clusters, each cluster having a different location on the segment map (310). The locations may be randomly selected. The locations may be even distributed across the transverse cross-section. The locations may be biased towards the centre of the segment map or may be biased towards another location of the segment map.

The quantity of clusters in the sample group may be freely chosen; the larger the quantity, the more the accurate the determination of target dose uncertainty.

A plurality of cluster target dose fractions (CTDFs) is determined (e.g. 50 to 200) for the plurality of clusters within the sample group.

FIG. 7A to 7D shows an example of a transverse cross-section (300) of the lumen at one candidate delivery location (130, a to c) and a sample group of multiple different clusters (306, a to d) (shaded lumen sections) each located at a different position on the segment map (310).

An uncertainty range for the candidate delivery location (130, a to c) may be determined, using the plurality of CTDF values assigned to clusters in the sample group.

From the sample group, a minimum CTDF and maximum CTDF may be determined, corresponding to the lumen segment providing the lowest CTDF and the lumen segment providing the highest CTDF, respectively. In addition, a median CTDF may be determined from the sample group. The minimum CTDF and maximum CTDF may be endpoints of an uncertainty range. The median CTDF may be a median of the CTDFs of the sample group. A CTDF distribution may be determined from the sample group.

The target dose uncertainty is related to the uncertainty range for the candidate delivery location (130, a to c).

For each candidate delivery location (130, a to c) the minimum CTDF, the maximum CTDF and the median CTDF may be determined as described above.

A difference between maximum CTDF and minimum CTDF may be termed ‘tip uncertainty’ (%). A candidate delivery location (130, a to c) having a lower tip uncertainty is preferred over a candidate delivery location (130, a to c) having a higher tip uncertainty.

The median CTDF may be termed the ‘effective target dose’ (%). The effective target dose is an indication of a mean likelihood of the dose reaching the target. A candidate delivery location (130, a to c) having a higher effective target dose is preferred over a candidate delivery location (130, a to c) having a lower effective target dose.

As mentioned elsewhere herein, the ‘tip uncertainty’ and/or ‘effective target dose’ are components of the target dose uncertainty.

The evaluating may comprise using the vascular cost and target dose uncertainty and optionally the coverage for each candidate delivery location (130, a to c). In other words, each candidate delivery location (130, a to c) is evaluated, based on the vascular cost and target dose uncertainty and optionally the coverage.

The evaluation (based on vascular cost and target dose uncertainty and optionally coverage), may lead to a score value. The target dose uncertainty may be a score value.

The tip uncertainty is preferably a scalar (score) value on a scale. The tip uncertainly is preferably a scalar (score) value on a scale having a lower limit and an upper limit (e.g. between 0 and 1, between 0 and 10, between 0 and 100). One end of the scale (e.g. the upper limit (1, 10, 100)) may be indicative of a high uncertainty that the tip will be disposed in a cluster for perfusion of the target (120); this might indicate a less suitable candidate delivery location. The other end of the scale (e.g. the lower limit (0)) may be indicative of a low uncertainty that the tip will be disposed in a cluster for perfusion the target (120)); this might indicate a more suitable candidate delivery location.

The effective target dose is preferably a scalar (score) value. The effective target dose is preferably a scalar (score) value on a scale having a lower limit and an upper limit (e.g. between 0 and 1, between 0 and 10, between 0 and 100). One end of the scale (e.g. the upper limit (1, 10, 100) may be indicative of a high fraction of a dose, released at the candidate delivery location, predicted to perfuse the target (120) (compared with non-target tissue); this might indicate a more suitable candidate delivery location. The other end of the scale (e.g. the lower limit (0)) may be indicative of low fraction of a dose, released at the candidate delivery location, predicted to perfuse the target (120) (compared with non-target tissue); this might indicate a less suitable candidate delivery location.

The target dose uncertainty may be a scalar (score) value. The target dose uncertainty may be a scalar (score) value on a scale having a lower limit and an upper limit (e.g. between 0 and 1, between 0 and 10, between 0 and 100). One end of the scale (e.g. the upper limit (1, 10, 100) may be indicative of a high uncertainty that the tip will be disposed in a cluster for perfusion the target (120) and a low effective target dose; this might indicate a less suitable candidate delivery location. The other end of the scale (e.g. the lower limit (0)) may be indicative of a low uncertainty that the tip will be disposed in a cluster for perfusion the target (120) and a high effective target dose; this might indicate a more suitable candidate delivery location.

The target dose uncertainty may be a scalar (score) value that is an average of the tip uncertainty and effective target dose. Tip uncertainty and effective target dose are each normalised according to a separate scale, wherein each scale has the same range (e.g. 0 to 1), and the same extremes of the scale represent the most favourable or most unfavourable locations.

The evaluation (based on vascular cost and Target dose uncertainty and optionally coverage and optionally radiation damage score) may be provided as one or more score values. The evaluation may be provided as a score value for vascular cost and a separate score value for Target dose uncertainty and optionally a separate score value for coverage. The evaluation may be provided as a combined score value for vascular cost and coverage and optionally coverage. The combined score is preferably an average (e.g. non-weighted, weighted) of individual scores for vascular cost and coverage and optionally coverage.

The substance delivered by the vascular catheter (200) at a candidate delivery location (130, a to c) may contain one or more radionuclides for treatment of the target (120). The one or more radionuclides can cause radiation damage to both non-target tissue (undesired) and to target tissue (desired for treatment). Accordingly, the method may further comprise determining, from a substance radiation score (SRS), a radiation damage score (RDS) for the candidate delivery location (130, a to c). A RDS is related to a likelihood of radiation damage caused by the one or more radionuclides in the substance to tissue of the organ (110). More in particular, a RDS is a likelihood that the substance flowing from the candidate delivery location (130, a to c) towards the target will cause damage to tissue of the target (120).

The RDS is a score, indicative of:
a likelihood that the substance flowing from the candidate delivery location (130, a to c) towards the target will cause damage to tissue of the target (120) of the organ (110), and/or
a likelihood that the substance flowing from the candidate delivery location (130, a to c) towards the target will cause damage to non-target tissue of the organ (110).

The likelihood of radiation damage in the target tissue and/or in the non-target tissue may be determined using the SRS and the target dose uncertainty, or methods used to calculate the target dose uncertainty.

As mentioned elsewhere herein the target dose uncertainty predicts a likelihood that the substance flowing from the candidate delivery location (130, a to c) towards the target tissue will flow through one or more arterial branches converging with the target, and into the target when delivered at the candidate delivery location (130, a to c). The likelihood that the substance flowing from the candidate delivery location (130, a to c) towards the non-target tissue will flow through one or more arterial branches converging with the target can be determined from the above target tissue prediction (e.g. 1 minus the likelihood in respect of the target tissue);

The coverage, or methods used to calculate the coverage, wherein the coverage predicts volume of target tissue contacted the substance delivered at the candidate delivery location (130, a to c) compared with total volume of the target, or the coverage predicts volume of non-target tissue contacted the substance delivered at the candidate delivery location (130, a to c) compared with total volume of the target.

A non-exhaustive list of radionuclides that may be present in the substance include one or more of Yttrium-90, Holmium-166, Lutetium-177, Rhenium-75.

Each radionuclide has a property profile which includes values for properties such as half-life, yield (quantity of therapeutic radiation emitted per unit of time), maximum tissue penetration depth, and the like. The property profile of a radionuclide is known in the art and/or can be measured by methods known in the art. The property profile may be a list of values, or may be a score generated using the values for the properties of the property profile.

Using the property profile of each radionuclide in the substance and the concentration (e.g. mmol/ml, or quantity of microspheres per ml) of each radionuclide in the substance a substance radiation score (SRS) may be generated for the substance. The SRS is preferably a scalar value. Typically, the higher the SRS, the higher damage the substance will cause to tissue.

As mentioned elsewhere herein, the SRS is used to determine the RDS for the candidate delivery location (130, a to c).

The RDS is preferably a scalar (score) value on a scale. The SRS is preferably a scalar (score) value on a scale having a lower limit and an upper limit (e.g. between 0 and 1, between 0 and 10, between 0 and 100).

One end of the scale (e.g. the upper limit (1, 10, 100)) may be indicative of a suitable delivery location according to damage caused to the tissue by the one or more radionuclides. For instance, a low likelihood of damage to the non-target tissue may contribute to an RDS at this end of the scale. For instance, a high likelihood of damage to the target tissue may contribute to an RDS at this end of the scale.

The other end of the scale (e.g. the lower limit (0) may be indicative of an unsuitable delivery location according to damage caused to the tissue by the one or more radionuclides. For instance, a high likelihood of damage to the non-target tissue may contribute to an RDS at this end of the scale. For instance, a low likelihood of damage to the target tissue may contribute to an RDS at this end of the scale.

The evaluating may comprise using the vascular cost and RDS, and optionally target dose uncertainty and optionally the coverage for each candidate delivery location (130, a to c). In other words, each candidate delivery location (130, a to c) is evaluated, based on the vascular cost and RDS, and optionally target dose uncertainty and optionally the coverage.

The evaluation (based on vascular cost and RDS, and optionally target dose uncertainty and optionally the coverage), may lead to a score value.

The evaluation (based on vascular cost and RDS, and optionally target dose uncertainty and optionally the coverage) may be provided as one or more score values. The evaluation may be provided as a score value for vascular cost and a separate score value for and RDS, and optionally a separate score value for target dose uncertainty and optionally a separate score value for the coverage. The evaluation may be provided as a combined score value for vascular cost and RDS, and optionally target dose uncertainty and optionally the coverage. The combined score is preferably an average (e.g. non-weighted, weighted) of individual scores for vascular cost and RDS, and optionally target dose uncertainty and optionally the coverage.

The evaluation may be based on a combination of vascular cost, coverage, target dose uncertainty (and where the substance contains one or more radionuclides, radiation damage score). The evaluation may be provided as one or more score values. The evaluation may be provided as a score value for vascular cost and a separate score value for Target dose uncertainty and a separate score value for coverage. The evaluation may be provided as a combined score value for vascular cost and coverage and Target dose uncertainty (and radiation damage score where applicable). The combined score is preferably an average of individual scores for vascular cost and coverage and Target dose uncertainty (and radiation damage score where applicable), or an average of weighted individual scores for vascular cost and coverage and Target dose (and radiation damage score where applicable).

The present method is performed in vitro. The present method is preferably performed ex vivo or in silico. The present method is performed using a computer. The present method is a computer-implemented method. The present method is performed offline. By offline, it is meant that the evaluating is performed on the three-dimensional model and three-dimensional model is received from a data storage device (e.g. hard-drive, cloud storage).

Further provided is a computing device or system configured for performing the method described herein.

Further provided is a computer program or computer program product having instructions which when executed by a computing device or system cause the computing device or system to perform (each of the steps of) a method as described herein.

Further provided is a computer readable medium having stored thereon a computer program (product) having instructions which when executed by a computing device or system cause the computing device or system to perform (each of the steps of) a method as described herein.

Further provided is a data stream which is representative of a computer program or computer program product having instructions which when executed by a computing device or system cause the computing device or system to perform (each of the steps of) the method as described herein.

The method may be performed using a computer system such as an Intel Architecture IA-32 based computer system 2 or using a supercomputing system, and implemented as programming instructions of one or more software modules stored on non-volatile (e.g. hard disk or solid-state drive) storage associated with the corresponding computer system. However, it will be apparent that at least some of the steps of any of the described processes could alternatively be implemented, either in part or in its entirety, as one or more dedicated hardware components, such as gate configuration data for one or more field programmable gate arrays (FPGAs), or as application-specific integrated circuits (ASICs), for example.

The method or system may produce an output that is:
displayed on a screen, or
saved to a file.

The present method of system may be regarded as a method for measuring or detecting a plurality of candidate delivery locations (130, a to c).

### EXAMPLE

A CT image dataset of the hepatic arterial vasculature of a hepatocellular carcinoma (HCC) subject was obtained by scanning the subject with a cone beam CT scanner (Philips Medical Systems, Netherlands) while intra-arterially injecting contrast agent into the left and right branches of the proper hepatic artery.

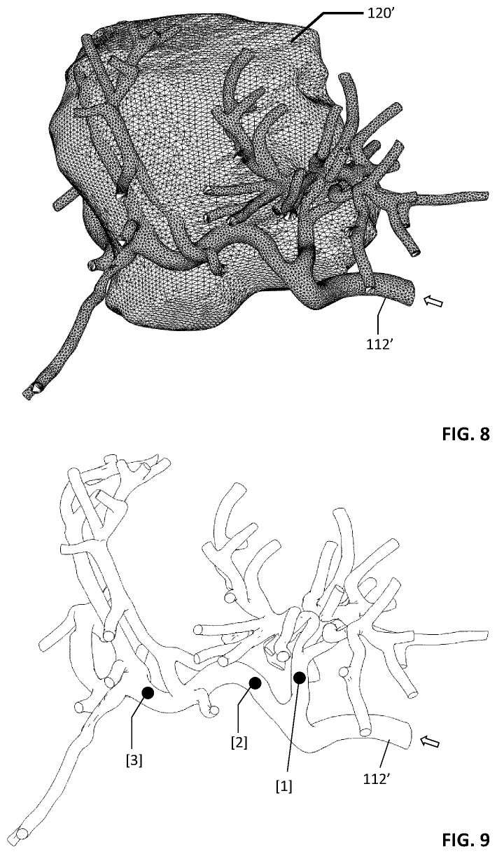

A 3D model was created from the 3D medical image. To create the 3D model, hepatic arteries were segmented in Mimics (Materialise, Belgium) based on the contrast difference between the arterial and venous trees in the arterial phase. A large tumour nodule (estimated volume: 310 ml) was identified. 3D model of the tumour mass (120′) and the hepatic arterial tree lumen (112′) (with 1 inlet at the proper hepatic artery level and 48 outlets) is depicted in FIG. 8. The hepatic arterial tree lumen (112′) is shown without the tumour mass (120′) in FIG. 9.

Three candidate delivery locations ([1], [2], [3]) were chosen from the 3D model for evaluation. The three locations ([1], [2], [3]) are shown in FIG. 9. Candidate delivery location [1] was in a proximal right hepatic artery (P-RHA), candidate delivery location [2] was in a proximal left hepatic artery (P-LHA), candidate delivery location [3] was in a distal right hepatic artery (D-RHA).

Different vascular cost parameters (D_{cdl}, κ_{cdl}, τ_{cdl}) were determined from the 3D model at each candidate delivery location ([1], [2], [3]). The value of the minimum diameter (D_{cdl}) of each candidate delivery location was determined as described herein. Tortuosity (τ_{cdl}) of each candidate delivery location was calculated as the deviation of the centerline from a straight line as described herein. Curvature κ_{cdl}) of each candidate delivery location centerline was calculated for each point i of the centerline, based on first and second derivatives of the centerline as described herein. A vascular cost (VC_{cdl}) for each candidate delivery location ([1], [2], [3]) was determined as a scalar (score) value that was an average of the normalised vascular cost parameters. The results are shown in Table 1 below. Candidate delivery location [1] having the lowest vascular cost was most preferred according to the vascular cost criterion alone.

TABLE 1
Comparison of different non-normalised vascular cost
parameters (D_{cdl}, κ_{cdl}, τ_{cdl}) and vascular cost (VC_{cdl}) at each
candidate delivery location. The vascular cost (VC_{cdl}) is
on a scale between 0 (most preferred) and 1 (least preferred).

| Candidate | Minimum | | | Vascular |
|---|---|---|---|---|
| delivery | diameter | Curvature | Tortuosity | cost |
| location | D_{cdl} | κ_{cdl} | τ_{cdl} | VC_{cdl} |
| [1] P-RHA | 4.6604 | 0.1395 | 0.0961 | 0.55 |
| [2] P-LHA | 6.2285 | 1.0081 | 0.1834 | 0.92 |
| [3] D-RHA | 4.6604 | 0.1523 | 0.1547 | 0.66 |

For each candidate delivery location, downstream hepatic artery outlets (downstream outlets) that perfuse the tumour were determined. A coverage (C) that was target coverage (TaC) of each candidate delivery location was determined as a percentage of the target (tumour) volume perfused by candidate delivery location compared with total tumour volume. To calculate the TaC, a region growing model was used (Vermijs et al., 2021, OncoDot.3 Symposium, Abstracts, 2021).

Downstream outlets present in the organ 3D model were selected as seed points. Hepatic arteries, tumour mass, and organ mass were included as boundaries. A perfusion zone was grown from each outlet, and outlets downstream of the candidate delivery location and within the tumour were selected. During region growing, voxels were added in the six orthogonal directions starting from each seed point. Growth was stopped when the volume of the tumour was filled. Region growing occurred simultaneously and at the same rate for all seed points. For each candidate delivery location, the corresponding downstream outlets were determined, and the corresponding perfusion zones were determined. For each candidate delivery location, a target grown volume (V_{tg}) was determined that was a summed total of corresponding perfusion zones grown within the tumour. A total target (tumour) volume (Vₜₜ) was measured from the 3D model.

For each candidate delivery location, target coverage (TaC) was determined as V_{tg}/Vₜₜ. The results are shown in Table 2 below.

TABLE 2
Comparison of target coverage at each candidate
delivery location. The target coverage (TaC) is on a scale
between 0 (least preferred) and 100 (most preferred).

| Candidate delivery location | Target coverage (TaC) (%) |
|---|---|
| [1] P-RHA | 69.6 |
| [2] P-LHA | 30.4 |
| [3] D-RHA | 52 |

With the catheter at the candidate delivery location [1] the target coverage was 69.6%; advancing the catheter to candidate delivery location [3] decreases the target coverage even further to 52%. Alternatively, injection candidate delivery location [2] leads to a target coverage of 30.4%. The full tumor might be targeted if two successive injections are performed in candidate delivery locations [1] and [2].

Target dose uncertainty was determined at each candidate delivery location ([1], [2] and [3]).

### 1^{st} Stage: Pre-Processing for Computational Fluid Dynamics (CFD)

Starting from the 3D model of the hepatic arteries, a computational mesh was created using finite-volume CFD software (Fluent by Ansys). The mesh density of the arterial body was determined through a mesh sensitivity analysis. Similarly, if a catheter is embedded in the hepatic arteries, a mesh sensitivity analysis of the catheter body will inform the mesh density of the catheter.

For each mesh element, discretized Navier-Stokes equations were resolved while advancing time steps to a pre-defined simulation end time. An inlet boundary condition, at the candidate delivery location, was a time-varying mass flow rate or velocity profile; outlet boundary conditions were mass flow fractions determined via the region growing model.

Behaviour of blood (modelling as a shear-thinning fluid) or a blood-mimicking fluid was one-way coupled with the behavior of the microparticles (inert microspheres or microbubbles).

### 2^{nd} Stage: Segment Map

A transverse cross-section was taken of the artery lumen at each of the candidate delivery locations ([1], [2] and [2]). The cross-section was divided into lumen segments of a segment map. In simulation, microparticles were released in a downstream direction for all the lumen segments of the segment map and tracked (Bomberna et al., Frontiers in Bioengineering and Biotechnology, 2022). Microparticles which escaped the domain through one of the downstream outlets were not tracked anymore; microparticles which developed no or incomplete trajectories within a pre-defined maximum number of steps, were rendered as ‘incomplete trajectories’.

### 3^{rd} Stage: Segment Target Dose Fraction

For each lumen segment of the segment map, a segment target dose fraction (STDF) was determined that was a ratio of the quantity of the microparticles delivered to the target from the lumen segment to the total quantity of microparticles released from the lumen segment.

### 4^{th} Stage: Cluster Target Dose Fraction

A cluster was generated from multiple adjacent lumen segments that together formed a shape of the catheter tip, and having a specific location on the segment map. For the cluster, a cluster target dose fraction (CTDF) was determined that was a ratio of the quantity of the microparticles delivered to the target from the cluster to the total quantity of microparticles released from the cluster.

### 5^{th} Stage: Sample Group Generation

Multiple (100) clusters and multiple corresponding cluster target dose fractions (CTDFs) were generated for a sample group. Each cluster had a different and random location on the segment map. A different sample group of 100 clusters was generated for each candidate delivery location ([1], [2], [3]).

### 6^{th} Stage: Uncertainty Range

For each candidate delivery location ([1], [2], [3]) a minimum CTDF, maximum CTDF and a median CTDF was determined for the sample group. The minimum CTDF and maximum CTDF were the endpoints of an uncertainty range.

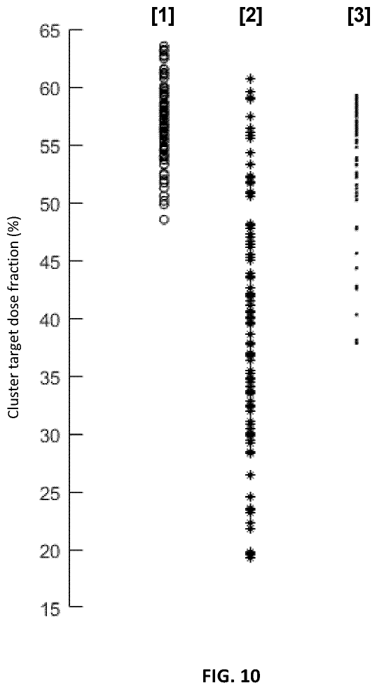

FIG. 10, shows, for each candidate delivery location ([1], [2], [3]), the CTDF within a sample group of 100 different clusters. The range of doses is largest for candidate delivery location [2], having the largest uncertainty range.

### 7^{th} Stage: Comparison of Effective Target Dose and Tip Uncertainty

For each candidate delivery location ([1], [2], [3]), target dose uncertainty, i.e. the effective target dose (%) (median TDF) and tip uncertainty (%) (difference between maximum and minimum TDF), were determined. In Table 3, the effective target dose and tip uncertainty were compared for the three candidate delivery location ([1], [2], [3]). For candidate delivery location [3], the effective target dose was roughly similar to that of candidate delivery location [1], but the tip uncertainty was significantly more uncertain (higher value). Additionally, for candidate delivery location [2], the effective target dose was much lower than for [1] and [3], and the uncertainty much higher than for [1] and [3], hence, candidate delivery location [2] was the least preferred choice based on target dose uncertainty. Based on the target dose uncertainty criterion alone, candidate delivery location [1] was the preferred choice.

TABLE 3
Target dose uncertainties for each candidate delivery location.
The effective target dose is on a scale between 0 (least preferred)
and 100 (most preferred). The tip uncertainty is on a scale
between 0 (most preferred) and 100 (least preferred).

| | Target dose uncertainty | |
|---|---|---|
| Candidate | Effective | Tip |
| delivery | target | uncertainty |
| location | dose (%) | (%) |
| [1] P-RHA | 57.0920 | 15.0240 |
| [2] P-LHA | 39.5060 | 41.2910 |
| [3] D-RHA | 57.1635 | 21.3850 |

The vascular cost, target coverage and target dose uncertainty were compared for each of the 3 candidate delivery locations. The full summary of the results is also given in Table 4.

TABLE 4
Collation of results of Tables 1 to 3.

| | | | | | | Target dose | |
|---|---|---|---|---|---|---|---|
| | | | | | Target | uncertainty | |
| Candidate | Minimum | | | Vascular | coverage | Effective | Tip |
| delivery | diameter | Curvature | Tortuosity | cost | (TaC) | target | uncertainty |
| location | D_{cdl} | κ_{cdl} | τ_{cdl} | (VC_{cdl}) | (%) | dose (%) | (%) |
| [1] P-RHA | 4.6604 | 0.1395 | 0.0961 | 0.55 | 69.6 | 57.0920 | 15.0240 |
| [2] P-LHA | 6.2285 | 1.0081 | 0.1834 | 0.92 | 30.4 | 39.5060 | 41.2910 |
| [3] D-RHA | 4.6604 | 0.1523 | 0.1547 | 0.66 | 52 | 57.1635 | 21.3850 |

Comments are made below concerning the results:
Selective injection in the P-RHA alone (69.6%) or P-LHA alone (30.4%) leads to target coverage significant less than 100%.
Vascular access in the P-LHA (VC_{cdl}=0.92) is significantly more difficult than in the P-RHA (VC_{cdl}=0.55) due to the high curvature region at the first bifurcation.
Injection in the P-LHA is associated with higher tip uncertainty (41.29%), and a lower effective target dose (39.51%) compared with the other candidate locations.
Comparing D-RHA and P-RHA, both the vascular access (VC_{P-RHA}=0.55; VC_{D-RHA}=0.66), and effective target dose (effective target dose_{P-RHA}=57.09%; effective target dose_{D-RHA}=57.16%;) are similar. However, for the D-RHA, the tip uncertainty is larger (tip uncertainty _{D-RHA}=21.39%; tip uncertainty _{P-RHA}=15.02%), and the target coverage is lower (target coverage_{D-RHA}=52%; target coverage_{P-RHA}=69.6%), showing little added value of super-selective over RHA injection.

An evaluation based on a combination of vascular cost (VC_{cdl}), coverage (target coverage (TaC)), and target dose uncertainty is the most informative:
Based only on vascular cost, candidate delivery locations P-RHA or D-RHA are preferable (similar cost) over the P-LHA. However, the lack of full target coverage for D-RHA injection is not considered.
Based on target coverage: a combination of candidate delivery locations D-RHA and P-LHA are preferred. However, the high vascular cost of P-LHA injection is not considered.
Based on Target dose uncertainty: candidate delivery locations P-RHA is optimal. However, the lack of full target coverage is not considered.

1. A computer-implemented method for evaluating a plurality of candidate delivery locations (130, a to c) for a delivery of a substance by a vascular catheter (200) for treatment of a target (120) within an organ (110) of a subject comprising:
receiving a three-dimensional model obtained from a three-dimensional medical image of at least a part of the organ (110) of the subject, wherein the three-dimensional model includes arterial lumens,
determining from the three-dimensional model a vascular cost, VC_{cdl}, for each candidate delivery location (130, a to c) which is related to an accessibility of the candidate delivery location (130, a to c) by a tip (202) of the vascular catheter (200),
determining, for each candidate delivery location (130, a to c) a target dose uncertainty, wherein:
the target dose uncertainty is a measure of a likelihood that the substance flowing from the candidate delivery location (130, a to c) towards the target will flow through one or more branches leading into the target (200), and
the target dose uncertainty is determined comprising the steps:
obtaining a segment map (310), wherein the segment map (310) is:
a transverse cross-section of an arterial lumen in the 3D model at the candidate delivery location (130, a to c); and
divided into a plurality of lumen segments (302, a to e);
predicting, for each candidate delivery location (130, a to c), a release of microparticles from the segment map (310) in a downstream direction, and a trajectory of each microparticle;
assigning to each lumen segment (302, a to e), a segment target dose fraction, STDF, value that is an indication of the quantity of microparticles released from that lumen segment (302, a to e) that flow into the target (120) compared with total quantity microparticles released from that lumen segment (302, m to p),
determining from the STDF values, a cluster target dose fraction, CTDF, wherein a cluster (306) is collection of lumen sections (302, b to e) grouped together to form a shape of a catheter tip (202) at a specific location on the segment map (310), and the CTDF is determined from the STDF values of lumen sections (302, b to e) within that cluster (306),
determining a plurality of the CTDF values for a sample group, wherein the each CTDF of the sample group is determined from a cluster having a different location on the segment map (310), and
determining from the sample group of CTDF values an uncertainty range for the candidate delivery location (130, a to c), and
determining the target dose uncertainty from the uncertainty range, wherein the evaluating comprises using a combination of the vascular cost and the target dose uncertainty for each candidate delivery location (130, a to c).

2. The computer-implemented method according to claim 1, wherein the vascular cost of a candidate delivery location (130, a to c) is determined from a set of vascular cost parameters of a portion (P) of the artery lumen, wherein the set of vascular cost parameters comprises at least one of:
minimum diameter, D_{cdl}, of the portion (P) of the artery lumen,
tortuosity, τ_{cdl}, of the portion (P) of the artery lumen,
curvature, κ_{cdl}, of the portion (P) of the artery lumen,
torsion, T_{cdl}, of the portion (P) of the artery lumen,
path length, PL_{cdl}, of the portion (P) of the artery lumen, and
branching angle, BA_{cdl}, of the portion (P) of the artery lumen,
wherein the portion (P) of the artery lumen has an end located at the candidate delivery location (130, a to c).

3. The computer-implemented method according to claim 2, wherein the set of vascular cost parameters comprises at least:
the minimum diameter, D_{cdl},
the tortuosity, τ_{cdl}, and
the curvature, κ_{cdl}.

4. The computer-implemented method according to claim 1, further comprising:
determining, for each candidate delivery location (130, a to e), a coverage, wherein the coverage is related to
target coverage (TaC) that is related to a volume of target (120) contacted by the substance when delivered at the candidate delivery location (130, a to c) by a tip of the vascular catheter (200) compared with total volume of the target (120), and
optionally non-target coverage (NTaC) that is related to a volume of non-target tissue within the organ contacted with the substance when delivered at the candidate delivery location (130, a to c) by the tip of the vascular catheter compared with total volume of the non-target tissue,
wherein the evaluating comprises using a combination of the vascular cost and the coverage for each candidate delivery location (130, a to c).

5. The computer-implemented method according to claim 4, wherein the target coverage (TaC) is determined by:
determining all downstream perfusion zones of the candidate delivery location (130, a to c), wherein each downstream perfusion zone is a three-dimensional region grown from a downstream outlet of the candidate delivery location (130, a to c), wherein the downstream outlet is an outlet of an artery lumen into the organ downstream of the candidate delivery location (130, a to c),
determining a target grown volume, V_{tg}, that is a summed total volume of downstream perfusion zones (134a1 to a5; 134b1 to b8; 134c1 to c4) for the candidate delivery location (130, a to c) disposed within the target (120),
determining a total target volume Vₜₜ, that is a total volume of the target, and
determining the target coverage comprising determining a ratio between V_{tg} and Vₜₜ.

6. The computer-implemented method according to claim 4, wherein the non-target coverage (NTaC) is determined by:
determining all downstream perfusion zones of the candidate delivery location (130, a to c), wherein each downstream perfusion zone is a three-dimensional region grown from a downstream outlet of the candidate delivery location (130, a to c), wherein the downstream outlet is an outlet of an artery lumen into the organ downstream of the candidate delivery location (130, a to c)
determining, a non-target grown volume, V_{ntg}, that is a summed total volume of perfusion zones (134a1 to a5; 134b1 to b8; 134c1 to c4) for the candidate delivery location (130, a to c) disposed in non-target tissue within the organ,
determining a total non-target volume Vₜₙₜ, that is a total volume of non-target, and
determining, the non-target coverage comprising determining a ratio between V_{ntg} and Vₜₙₜ.

7. The computer-implemented method according to claim 1, wherein the uncertainty range has one of more of:
a maximum CTDF value,
a minimum CTDF value, and
a median CTDF value.

8. The computer-implemented method according to claim 7, wherein the target dose uncertainty comprises a tip uncertainty component, and wherein the tip uncertainty is
indicative of an uncertainty that the catheter tip (202) would be disposed in a cluster causing flow of substance into the target (120), and
determined comprising taking difference between the maximum CTDF value and the minimum CTDF value.

9. The computer-implemented method according to claim 7, wherein the target dose uncertainty comprises an effective target dose component, and wherein a high effective target dose is
indicative of a proportion of the substance that is expected to reach the target (120), and
determined comprising taking the median CTDF value.

10. The computer-implemented method according to claim 1, further comprising:
determining, for each candidate delivery location (130, a to c) a radiation damage score, wherein
the substance contains one or more radionuclides; and
the radiation damage score is a likelihood that the substance flowing from the candidate delivery location (130, a to c) towards the target will cause damage to tissue of the target (120);
wherein the evaluating comprises using a combination of the vascular cost and the radiation damage score for each candidate delivery location (130, a to c).

11. The computer-implemented method according to claim 7, wherein the evaluation of a candidate delivery location (130, a to c) comprises using a combination of the vascular cost, the coverage, the target dose uncertainty and the radiation damage score for the candidate delivery location (130, a to c).

12. A computing device or system configured for performing the method according to claim 1.

13. A computer program or computer program product having instructions which when executed by a computing device or system cause the computing device or system to perform the method according to claim 1.

14. A data stream which is representative of a computer program or computer program product having instructions which when executed by a computing device or system cause the computing device or system to perform the method according to claim 1.
